(19) United States
(12) Patent Application Publication
(10) Pub. No.: US 2013/0315779 A1
(43) Pub. Date: Nov. 28, 2013

(54) METHOD FOR CONTROLLING ODORS ASSOCIATED WITH ANIMAL AND PLANT BYPRODUCTS

(71) Applicant: Contact Marketing Solutions Innovative Technologies, Danbury, CT (US)

(72) Inventors: David H. Creasey, Splendora, TX (US); Jason H. Creasey, Conroe, TX (US); Jerry B. Creasey, Conroe, TX (US)

(21) Appl. No.: 13/798,623

(22) Filed: Mar. 13, 2013

Related U.S. Application Data
(60) Provisional application No. 61/651,259, filed on May 24, 2012.

(51) Int. Cl. A61L 11/00; A01N 59/26; A01N 59/20; A01N 59/02

(52) U.S. Cl. CPC A61L 11/00; A01N 59/02; A01N 59/26; A01N 59/20
USPC 422/5; 424/703; 424/710; 424/605; 424/637

(57) ABSTRACT

An aqueous, acidic composition and method for controlling odors associated with animal and plant processing and for controlling bacterial growth is presented.

### RELATED APPLICATIONS

This application claims priority to U.S. Provisional Application No. 61/651,259, which was filed on May 24, 2012, and is incorporated herein by reference, in entirety, for all purposes.

### FIELD OF THE INVENTION

The invention relates generally to a composition and method for controlling odors arising from animal and plant processing and for controlling bacterial growth.

### BACKGROUND OF THE INVENTION

Rendering, the process that converts waste animal tissue into stable, value-added materials, presents a high degree of complexity relating to volatile organic compounds (VOCs). Some of the major emissions related to rendering include organic sulfides, disulfides, c-4 to c-7 aldehydes, trimethylamine, c-4 amines, quinolone, dimethyl pyrazine, other pyrazines c-3 to c-6 organic acids, c-7 alcohols, and aliphatic hydrocarbons. The suppression and/or termination of the complex VOCs of a rendering operation is a costly and difficult problem to correct by conventional means.

There is a need for improved, cost-effective and environmentally friendly methods to control odors associated with rendering of animal tissue and also processing of plant materials. Such a method would allow the rendering material to be transported greater distances to rendering plants for processing and to be held for longer periods of time.

### SUMMARY OF THE INVENTION

A composition for controlling odors associated with plant and animal processing is presented. The composition comprises a first component consisting of an acid selected from the group consisting of sulfuric acid, phosphoric acid, fumaric acid, acetic acid, nitric acid, and hydrochloric acid; a second component consisting of at least one of an ammonium compound, magnesium sulfate, potassium sulfate, and sodium sulfate; a cyclodextrin; and at least one metal ion selected from the group consisting of copper, zinc, magnesium, manganese, nickel, iron, titanium, and the noble metals, wherein the metal ion is provided as a metal sulfate.

Methods for preparing and using the composition are also described. The composition may be prepared by (a) preparing an aqueous, concentrated acidic solution by the steps comprising
i) combining in a pressurized or non-pressurized vessel an acid of at least 89% purity, selected from the group consisting of sulfuric acid, phosphoric acid, fumaric acid, acetic acid, nitric acid, and hydrochloric acid with water and at least one of an ammonium compound, magnesium sulfate, potassium sulfate, and sodium sulfate to provide a mixture;
ii) maintaining the temperature of the mixture of step i) in a range from about 125° F. to about 1000° F. and allowing the components of the mixture to react for a selected time;
iii) cooling the mixture or allowing the mixture to cool;

(b) diluting the mixture with water;

(c) adding a selected amount of a cyclodextrin and a selected amount of at least one metal ion to the solution of step (b).

The composition may be used for controlling odors associated with animal and plant processing by the steps comprising diluting the composition and applying the diluted solution to a surface, space, or body of water used for animal, plant, or food processing. The composition may be used for controlling bacterial growth by the steps comprising diluting the composition and applying the diluted solution to a surface, space, or body of water.

### BRIEF DESCRIPTION OF THE DRAWINGS

FIG. 1 is a graph of the effects of chlorine, water, and the test solution on odor intensity associated with chicken renderings over time. The study is described in Example 1.

### DETAILED DESCRIPTION OF THE INVENTION

An acidic composition of matter is disclosed in U.S. Pat. Nos. 5,989,595 and RE 41,109 to Cummins, which are incorporated herein by reference in their entirety. As described below, it has now been found that a modification of this composition is useful for controlling odors associated with processing animal and plant products, e.g., in animal rendering and food processing facilities, and for controlling bacterial growth.

The composition is aqueous and comprises, as a first component, an acid selected from sulfuric acid, phosphoric acid, fumaric acid, acetic acid, nitric acid or hydrochloric acid. The acid is preferably of high purity, i.e., between about 89% to about 99.9% purity. A second component is at least one of an ammonium compound, sodium sulfate, potassium sulfate, and magnesium sulfate. The ammonium compound is preferably anhydrous ammonia, ammonia monohydrate, ammonium sulfate, or urea ammonium nitrate (UAN), most preferably, ammonium sulfate. The composition further comprises at least one metal ion. The metal ion can include, but is not limited to, copper, zinc, magnesium, manganese, nickel, iron, titanium, and the noble metals, e.g., gold, silver, platinum, palladium. The metal ion is generally provided as a metal sulfate.

In one embodiment, the second component of the composition is prepared by first heating water of any type, preferably distilled or deionized water, to between about 65° F. and about 210° F., preferably about 70° F. to about 170° F. The ammonium compound is added to the heated water to give a concentration of 5% to 35% and the solution is mixed to dissolve the ammonium compound. In other embodiments, sodium sulfate, potassium sulfate, and/or magnesium sulfate is combined with the water instead of or in addition to an ammonium compound. In another embodiment, the second component is added to water that has not been heated.

The resulting solution of the second component is then added simultaneously with the acid to a pressure vessel. The acid is added to comprise about 10% to about 60%, preferably about 15% to about 40%, of the final concentrated solution. In one embodiment, the reaction is allowed to proceed under pressure in the range of atmospheric to about 80 psi above atmospheric pressure, preferably between atmospheric and 15 psi above atmospheric pressure, while a DC current is passed through the mixture at a current of at least one amp. In other embodiments the addition of pressure and/or DC current is omitted.

The time and temperature of the reaction will vary based on the amount of reactants, size of reactor and reactivity of selected reactants. The temperature of the mixture is maintained in a range between about 125° F. and about 1000° F. during the reaction. The reaction time varies between about 30 minutes and about 6 hours, preferably between 1 and 3 hours. A cooling jacket is required to control the temperature of the reaction. After the reaction has proceeded to completion, the mixture is allowed to cool or can be cooled by any appropriate means. In one embodiment, the original solution of the second component is added at about 10% to about 15% of the total weight of the final, cooled mixture. In another embodiment, this step is omitted.

The pH of the final, concentrated solution is zero or less. The concentrated solution is diluted in a range of from 1:10 to 1:5000 with water, preferably distilled or deionized water, and additional components are added.

One or more metal sulfates are added to the solution in a concentration range of 0.5% to 30%. One or more other components can be added to the diluted solution to enhance its performance in controlling odors associated with processing animal and plant materials. For example, microporous aluminosilicate minerals may be added to the solution at 0.5 g/L to 50 g/L, preferably 5.0 g/l to 25 g/L.

A polyol, such as propylene glycol, glycerine, guar gum, etc., can be added at 2 to 80 ml/L. Sodium acid pyrophosphate (SAPP) can be added in a concentration range of about 1% to about 8%, preferably about 3% to about 5%. Phosphoric acid, preferably about 75% grade, can be added in a concentration range of 2 to 20 ml/L preferably 5 to 15 ml/L. A cyclodextrin, preferably alpha, beta, or gamma cyclodextrin, most preferably beta cyclodextrin, can be added to a final concentration of 2 to 20 g/L, preferably 6 to 12 g/L. Mixing of the components can be performed by any appropriate means.

Preservatives, such as sodium benzoate and potassium benzoate, may also be added to the composition to deter fungal growth. Sodium or potassium benzoate may be added in a concentration range of 0.05% to 10%.

To reduce odors or control bacterial growth, the diluted solution is applied to a surface by any appropriate method, e.g., spraying, pouring, spreading with a cloth, sponge, etc. The solution can also be applied to the air in a space or to a body of water, e.g., waste water ponds. The solution can be used on most natural and man-made surfaces including, but not limited to, metal, plastic, glass, concrete, fabric, carpet, rubber, vinyl, polymeric, stone, earth, and wood surfaces.

### EXAMPLES

### 1. Control of Odors Associated with Chicken Renderings

Chicken renderings (offal) were obtained from a chicken processing plant and distributed among plastic containers with lids. Each container held 4 ounces of chicken renderings. Spray bottles were used to spray each container with about 2 ml of a test or control solution. The test solution was a 1:20 dilution of the concentrate prepared as described above with sulfuric acid and ammonium sulfate, and also containing 5 g/L phosphoric acid, 4 wt % SAPP, 8 g/L beta cyclodextrin, 3.6 ml/L propylene glycol and 135 ppm copper sulfate. Control solutions were chlorine bleach (0.2 g/L) and water. Experiments with the test solution and chlorine bleach were repeated 10 times. Experiments with water were repeated 5 times. The experiments were performed at ambient temperatures of about 83° F. to about 88° F., under closed lid conditions, i.e., container lids remained in place throughout the total data collection time.

Odor from each container was measured by a handheld odor meter (Shinyei OMX-SR) before spraying, after spraying, and at periodic intervals up to 9 hours.

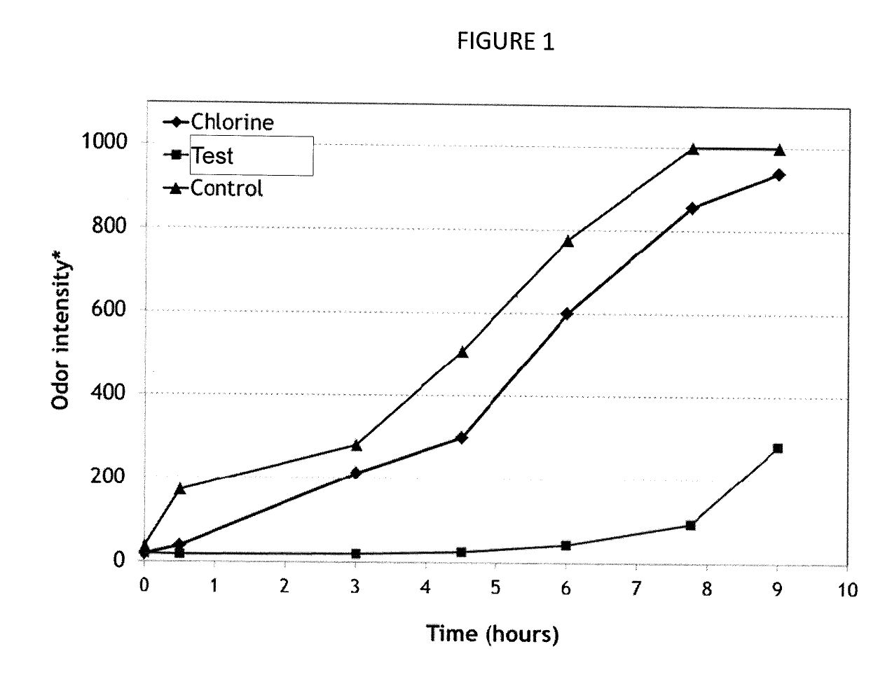

Results are shown in Table I and in FIG. 1 as the mean odor intensity for replicate experiments. The test solution provided significantly greater odor reduction than the control solutions over the 9 hours tested (p<0.05).

TABLE 1

| | Chlorine | | | Control | | | Test solution | | |
|---|---|---|---|---|---|---|---|---|---|
| | | | Tukey- | | | Tukey- | | | Tukey- |
| Time | | | Kramer | | | Kramer | | | Kramer |
| (h) | MOI | ±SD | HSD* | MOI | ±SD | HSD* | MOI | ±SD | HSD* |
| 0 | 18 | 9 | e | 38 | 17 | de | 20 | 7 | e |
| 0.5 | 38 | 15 | e | 172 | 131 | de | 17 | 6 | e |
| 3 | 213 | 207 | de | 281 | 150 | cde | 20 | 11 | e |
| 4.5 | 300 | 188 | cd | 507 | 212 | bc | 25 | 10 | e |
| 6 | 600 | 282 | b | 776 | 132 | ab | 43 | 26 | e |
| 7.75 | 856 | 217 | a | 999 | 0 | a | 92 | 49 | e |
| 9 | 939 | 155 | a | 999 | 0 | a | 281 | 160 | cd |

MOI—mean odor intensity
*Means followed by the same letter are not significantly different (p < 0.05).

Mean odor intensity averaged over the 9 hour period was significantly lower for the test solution (71.3 MOI) compared with chlorine (423.5 MOI) and control (water) (539 MOI). Mean MOI over 9 hours was not significantly different between chlorine and water controls.

### 2. Control of Bacterial Growth

The test solution described in Example 1 was tested against bacterial growth using a modification of AOAC method 960.09. Sterile test tubes containing the test solution or control (water) were inoculated at an inoculum level of about 1-5×10⁶ colony forming units (CFU)/mL per tube of Salmonella, E. coli, or Listeria monocytogenes. Five strains of each bacterial type were tested. Duplicate tubes were sampled at 15 minutes, 1 hour or 24 hours after inoculation. Sample aliquots were diluted and plated on tryptic soy agar plates. Plates were incubated for 24-48 h at 35° C. Inoculum counts were expressed as CFU/ml and converted to log₁₀ transforms. Log₁₀, unit reductions versus the control sample were calculated for each inoculum/dilution/time combination. Results are shown in Table 2.

TABLE 2

| | Salmonella | | | Listeria | | | E. coli | | |
|---|---|---|---|---|---|---|---|---|---|
| | Log₁₀ CFU/ml* | | | Log₁₀ CFU/ml | | | Log₁₀ CFU/ml | | |
| | Con- | | | Con- | | | Con- | | |
| Time | trol | Test | LR** | trol | Test | LR | trol | Test | LR |
| 15 min | 6.03 | <0.70 | >5.3 | 5.91 | <0.70 | >5.2 | 5.36 | 1.26 | 4.1 |
| 1 h | 6.54 | <0.70 | >5.8 | 5.98 | <0.70 | >5.3 | 6.03 | 2.0 | 4.0 |
| 24 h | 6.59 | <0.70 | >5.9 | 5.61 | <0.70 | >4.9 | 6.40 | <0.70 | >5.7 |

*Log₁₀ CFU/ml is mean of two repetitions
**LR is log reduction = (log unit count of control) − (log unit count of test)

A log reduction of at least 4.1 was demonstrated for treatment with the test solution of all three bacterial species within 15 minutes of treatment. Salmonella, Listeria, and E. coli counts were reduced by the test solution at all three time periods by at least 99.9%. Additional analyses demonstrated that the test solution also reduced growth of Clostridium sp. by 99.7% after 5 h of treatment (data not shown). Because the composition kills bacteria that consume the original waste material, treatment with the composition should also increase the protein concentration of the processed material.

We claim:

1. A composition for controlling odors comprising a first component consisting of an acid selected from the group consisting of a sulfuric acid, phosphoric acid, fumaric acid, acetic acid, nitric acid and hydrochloric acid; a second component consisting of at least one of an ammonium compound, magnesium sulfate, potassium sulfate, and sodium sulfate; a cyclodextrin; and at least one metal ion selected from the group consisting of copper, zinc, magnesium, manganese, nickel, iron, titanium, and the noble metals, wherein the metal ion is provided as a metal sulfate.

2. The composition of claim 1, wherein the ammonium compound is selected from the group consisting of anhydrous ammonia, ammonia monohydrate, ammonium sulfate, and urea ammonium nitrate.

3. The composition of claim 1, further comprising a polyol.

4. The composition of claim 1, further comprising sodium acid pyrophosphate.

5. The composition of claim 1, further comprising phosphoric acid.

6. The composition of claim 1, wherein the composition comprises sulfuric acid, ammonium sulfate, copper sulfate, and beta cyclodextrin.

7. The composition of claim 6, further comprising phosphoric acid and sodium acid pyrophosphate.

8. The composition of claim 1, wherein the composition comprises a metal ion selected from copper, zinc, and silver.

9. The composition of claim 1, wherein the cyclodextrin is selected from the group consisting of alpha cyclodextrin, beta cyclodextrin, and gamma cyclodextrin.

10. A method for controlling odors associated with animal and plant processing comprising diluting the composition of claim 1 and applying the diluted solution to a surface, space, or body of water used for animal, plant, or food processing.

11. A method for controlling bacterial growth on a surface or in a body of water comprising diluting the composition of claim 1 and applying the diluted solution to the surface or body of water.

12. The method of claim 10, wherein the diluted solution is applied by spraying.

13. A method for preparing the composition of claim 1 comprising the steps of
(a) preparing an aqueous, concentrated acidic solution by the steps comprising
i) combining in a pressurized or non-pressurized vessel an acid of at least 89% purity, selected from the group consisting of sulfuric acid, phosphoric acid, fumaric acid, acetic acid, nitric acid, and hydrochloric acid with water and at least one of an ammonium compound, magnesium sulfate, potassium sulfate, and sodium sulfate to provide a mixture;
ii) maintaining the temperature of the mixture of step i) in a range from about 125° F. to about 1000° F. and allowing the components of the mixture to react for a selected time;
iii) cooling the mixture or allowing the mixture to cool;
(b) diluting the mixture with water;
(c) adding a selected amount of a cyclodextrin and a selected amount of at least one metal ion to the solution of step (b).

14. The method of claim 13, wherein the at least one metal ion is selected from the group consisting of copper, zinc, magnesium, manganese, nickel, iron, titanium, and the noble metals, wherein the metal ion is added as a metal sulfate.

15. The method of claim 14, wherein the metal sulfate is selected from copper sulfate, zinc sulfate, and silver sulfate.

16. The method of claim 15, wherein the metal sulfate is copper sulfate and the copper sulfate is added to the solution in a range from 0.5 to 30%.

17. The method of claim 13, wherein the ammonium compound is selected from the group consisting of anhydrous ammonia, monohydrate ammonia, ammonium sulfate, and urea ammonium nitrate.

18. The composition of claim 1, further comprising sodium benzoate or potassium benzoate.

19. The composition of claim 1, further comprising a microporous aluminosilicate mineral.
